(19) United States
(12) Patent Application Publication
(10) Pub. No.: US 2013/0092563 A1
(43) Pub. Date: Apr. 18, 2013

(54) CONDOM CONTAINER WITH REMOVABLE CARRYING CASE

(71) Applicant: Global Protection Corporation, Boston, MA (US)

(72) Inventors: Davin Thomas Wedel, Boston, MA (US); Jared Thomas Maraio, Dorchester, MA (US)

(73) Assignee: Global Protection Corporation, Boston, MA (US)

(21) Appl. No.: 13/645,074

(22) Filed: Oct. 4, 2012

Related U.S. Application Data
(60) Provisional application No. 61/543,703, filed on Oct. 5, 2011.

(51) Int. Cl. A61F 6/00; B65B 7/28

(52) U.S. Cl. CPC A61F 6/005; B65B 7/28
USPC 206/69; 53/467

(57) ABSTRACT

The invention relates to a container for holding at least one prophylactic device. The container includes a canister that has an open and a closed end and that is sized to store several prophylactic devices. The container also includes a first carrying case portion and a second carrying case portion. The first carrying case portion is configured to fit together with the second carrying case portion in order to form a carrying case for at least one, but fewer than all, of the prophylactic devices from the canister. The canister, the first carrying case portion, and the second carrying case portion are configured such that the first and second carrying case portions can be securely, but removably fixed to the canister.

### RELATED APPLICATIONS

This application claims the benefit of U.S. Provisional Application No. 61/543,703, filed Oct. 5, 2011. The specifications of each of the foregoing applications are incorporated herein by reference in their entirety.

### FIELD OF THE INVENTION

The present invention relates to a container for carrying prophylactic devices. Specifically, the invention relates to a container comprising a canister and two carrying case portions, wherein the carrying case portions may be fitted together to form a carrying case.

### BACKGROUND OF THE INVENTION

Before entering the stream of commerce, prophylactic devices, such as condoms, are traditionally wrapped individually and then packaged and sold together in a small container, often a cardboard box. In the case of condoms, such packaging is useful for storing at one's home, but when one wishes to carry only one or two condoms outside the home, the average purchaser carries the condom or condoms in his or her wallet, purse, pocket or luggage. However, such storage practices are prone to causing damage to the condom itself, thereby potentially eliminating the utility of the condom as a prophylactic device. For example, as one tends to store other objects, such as coins, keys, pens or pencils, in one's wallet, purse, pocket or luggage, a condom stored in any one of these locations could experience a pressure against such objects that is strong enough to burst or puncture the condom, or at least the wrapper, resulting in degradation of the condom. Also, as one tends to routinely remove and disturb objects stored in these locations, it is foreseeable that a small condom package stored in these locations could get lost or misplaced, and therefore not be available when desired.

### SUMMARY OF THE INVENTION

It would be useful to provide a container that is filled with one or multiple prophylactic devices, e.g., condoms, and that also includes a sturdy carrying case that can transport or store one or multiple prophylactic devices.

In one embodiment, the invention relates to a container for holding at least one prophylactic device. In this embodiment, the container includes a canister that has a first end and a second end and that is sized to store several prophylactic devices. In this embodiment, the container also includes a first carrying case portion and a second carrying case portion. The first carrying case portion is configured to fit together with the second carrying case portion in order to form a carrying case for at least one, but fewer than all, of the prophylactic devices from the canister. Also in this embodiment, the canister, the first carrying case portion and the second carrying case portion are configured such that the first and second carrying case portions can be securely, but removably, fixed to the canister.

In some embodiments, the container includes a lid that is configured to engage both an open end of the canister and the first carrying case portion. With this lid, the first carrying case portion may be securely, but removably, attached to the canister. In other embodiments, the first carrying case portion is configured to directly engage and at least partially close the open end of the canister. In some embodiments, the second carrying case portion is configured to engage a closed end of the canister. In other embodiments, both ends of the canister are open.

In some embodiments, the first and second carrying case portions are each threaded such that they can be can be screwed together. In other embodiments, the first and the second carrying case portion engage to form a hinge at a first position and a clasp at a second position. In some embodiments, the first carrying case portion has a hinged portion capable of being opened in order to access a prophylactic device held within the carrying case. In some embodiments, the carrying case is pocket-sized.

In some embodiments, the prophylactic device stored in the container is a condom. In some embodiments, the container includes a divider that is configured for insertion into the carrying case and that separates the carrying case into a first portion sized to hold a condom and a second portion sized to hold a second object. In some embodiments, the second object is a package of lubricant or a packaged dental dam.

In some embodiments, the container also includes a series of linked pockets. In these embodiments, the series of linked pockets is sized to fit within the carrying case. Also in these embodiments, each pocket is sized to accommodate a condom.

In some embodiments, the first carrying case portion has a lip over which the second carrying case portion slides in order to engage the first carrying case portion. In some embodiments, the first carrying case portion has an indentation at a location that, when the first carrying case portion is engaged with the second carrying case portion, facilitates a user applying a separation pressure to the second carrying case portion to disengage the first carrying case portion from the second carrying case portion.

In some embodiments, the canister has a circular, hexagonal, rectangular or oval cross-section. In some embodiments, the canister has a primary axis and is shaped to store multiple prophylactic devices stacked along the primary axis. In certain embodiments, the canister stores at least five times the number of individual prophylactic devices that can fit in the carrying case. In some embodiments, the canister is transparent.

According to another aspect, the invention relates to a method for packaging condoms. This method includes the steps of filling a canister having a first end and a second end with multiple condoms. The method also includes the step of securely, but removably, fixing a first carrying case portion to the first end of the canister and securely, but removably, fixing the second carrying case portion to the second end of the canister. The carrying case portions used in this method are also configured to engage each other to form a carrying case. At least one, but fewer than all, of the condoms filled into the canister may be stored in the carrying case. In some embodiments, the method also includes the step of shrink-wrapping the canister along with the first and second carrying case portions securely but removably attached to it.

### DESCRIPTION OF THE DRAWINGS

The invention may be better understood from the following illustrative description with reference to the following drawings.

FIG. 1 is a side and perspective view of one embodiment of the container of the present invention.

FIG. 2 is an exploded view of two different embodiments of the container from FIG. 1. In panel A, an optional lid is included as a component of the container. In panel B, the optional lid is not present as a component of the container.

FIG. 3 is an exploded view (panel A) and a side view (panel B) of one embodiment of the canister from FIGS. 1 and 2. In this embodiment, the canister holds multiple condoms.

FIG. 4 depicts several different embodiments of the invention. Containers having oval (panel A), rectangular (panel B), square (panel C) or hexagonal (panel D) cross-sections are depicted. In these embodiments, the containers do not include the lids.

FIG. 5 depicts one embodiment of the invention, where the container has a first carrying case portion and a second carrying case portion that are each fitted to plastic molding on the sides of the canister. The bolded arrows indicate that the first and second carrying case portions may be fitted to the plastic molding on the canister and also easily removed.

FIG. 6 depicts the first and second carrying case portions from FIGS. 1 and 2. Panel A depicts the carrying case portions fitted together to form a carrying case. Panel B depicts an exploded view of the first and second carrying case portions and several condoms capable of being stored within the carrying case.

FIG. 7 depicts an embodiment of the container where the carrying case portions from FIGS. 1 and 2 may be hinged together.

FIG. 8 depicts several different embodiments of the container in which one of the carrying case components from FIGS. 1 and 2 has a separate compartment for storing objects that are not condoms. Panels A-B depict a removable divider over which a condom may be stored and under which a package of lubricant or a packaged dental dam may be stored or visa-versa. Panels C-D depict a divider that divides the cross-section of one of the carrying case portions such that a condom or condoms may be stored on one side of the container and either a package of lubricant (panel C) or a packaged dental dam (panel D) is stored on the other side of the carrying case.

FIG. 9 depicts the carrying case from FIG. 7 having a hinged door on the second carrying case portion which may be opened to remove a condom.

FIG. 10 depicts the carrying case from FIG. 7 having an indentation just below the point where the first carrying case portion meets the second carrying case portion.

FIG. 11 depicts the carrying case from FIG. 7 having threading on the lip portion of the first carrying case portion.

FIG. 12 depicts the carrying case from FIG. 7 having a collapsible series of pockets for storing multiple condoms.

FIG. 13 is a flow chart of a method of packaging condoms, according to an illustrative embodiment of the invention.

FIG. 14 depicts an alternative embodiment of the container, which includes two canisters, according to an illustrative embodiment of the invention.

### DETAILED DESCRIPTION OF THE INVENTION

To provide an overall understanding of the invention, certain illustrative embodiments will now be described. However, it will be understood by one of ordinary skill in the art that the containers and methods described herein may be adapted and modified as is appropriate for the application being addressed and that the containers and methods described herein may be employed in other suitable applications, and that such other additions and modifications will not depart from the scope thereof.

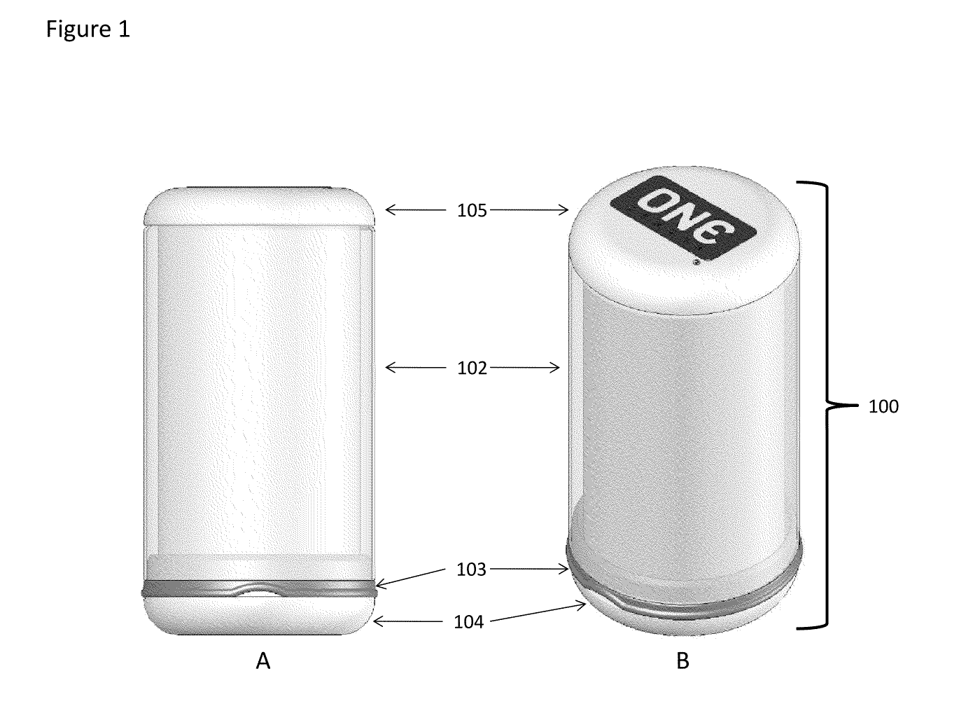

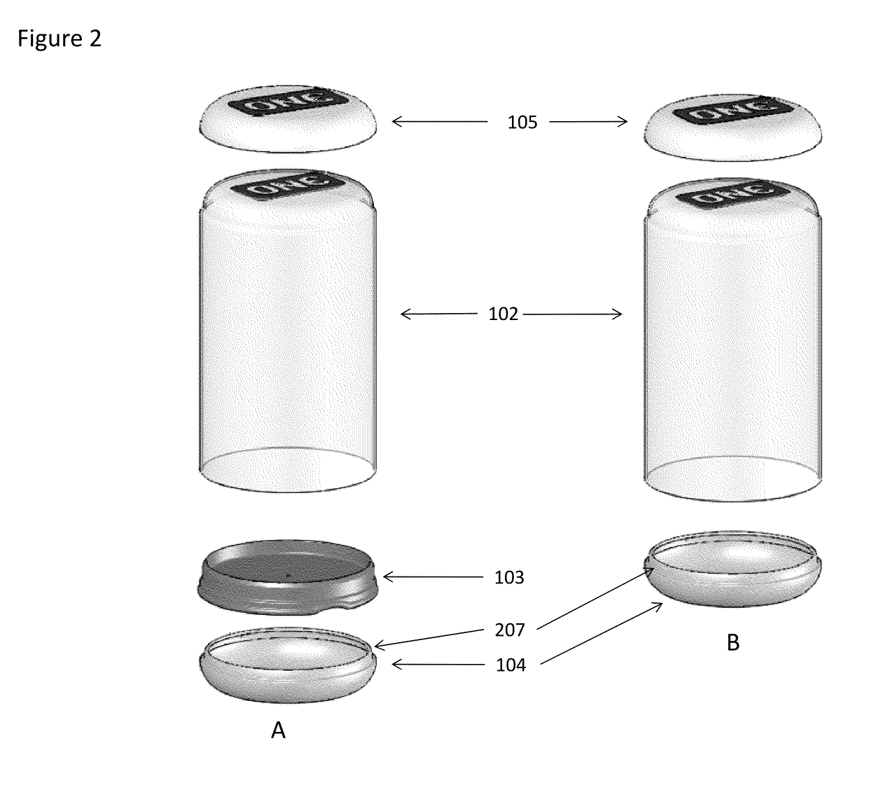

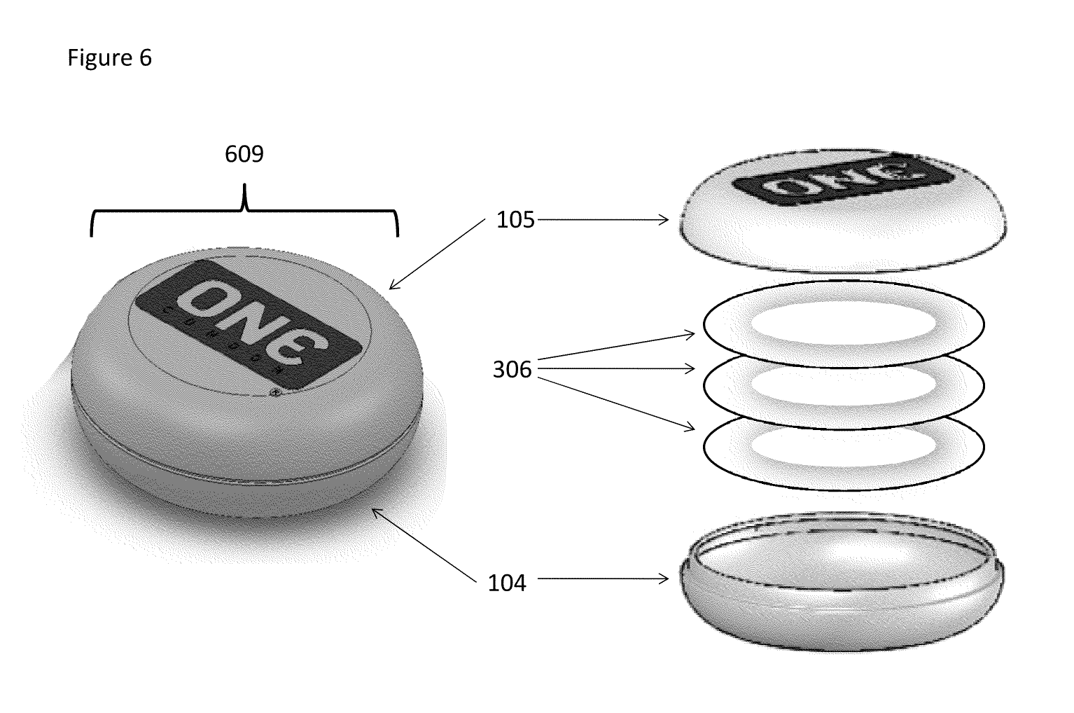

FIGS. 1 and 2 illustrate various embodiments of a container 100 for distributing prophylactic devices, such as condoms. The container 100 includes a canister 102 having an open end and a closed end. In this case, the container 100 also includes a lid 103, which can directly engage the open end of the canister 102. The container 100 also includes a first carrying case portion 104, which can directly engage the lid 103, and a second carrying case portion 105, which can directly engage the closed end of the canister 102. In certain embodiments (FIG. 2, panel B), the container does not include a lid 103, and, instead, the first carrying case portion 104 may be fixed securely, e.g., by means of a lip 207 to the canister 102. As illustrated in FIG. 6, the first carrying case portion 104 and the second carrying case portion 105 also can be separated from the canister 102 and fitted together to form a carrying case 609. In one embodiment, the carrying case portions 104 and 105 are formed form a light weight metal, such as aluminum or tin or a metal alloy. In alternative embodiment, the carrying case portions 104 and 105 are formed from plastic, for example, through an injection molding process.

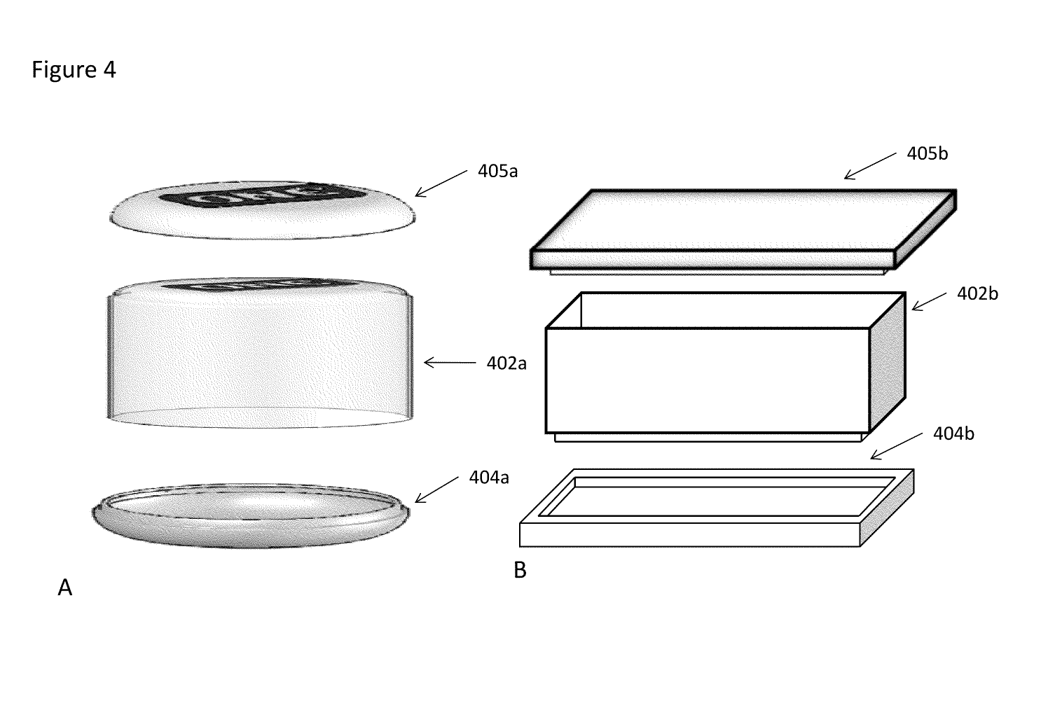

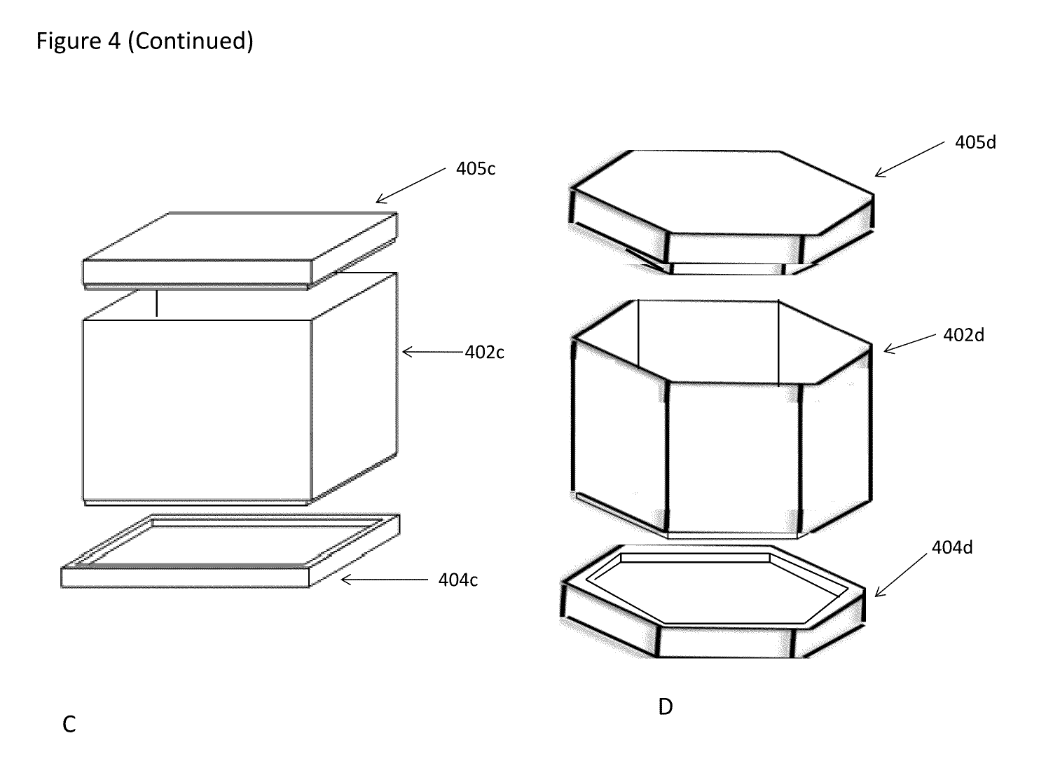

The canister 102 and carrying case 609 may be any shape that accommodates the storage of at least one prophylactic device. As illustrated in FIGS. 1 and 2, the first carrying case portion 104, canister 102, lid 103, and second carrying case portion 105, may have round cross-sections. As illustrated in FIG. 4, panel A, the first carrying case portion 404a, canister 402a, and second carrying case portion 405a, may have oval cross-sections (FIG. 4, panel A). As illustrated in FIG. 4, panel B, the first carrying case portion 404b, canister 402b, and second carrying case portion 405b, may have rectangular cross-sections (FIG. 4, panel B). As illustrated in FIG. 4, panel C, the first carrying case portion 404c, canister 402c, and second carrying case portion 405c, may have square cross-sections (FIG. 4, panel C). As illustrated in FIG. 4, panel D, the first carrying case portion 404d, canister 402d, and second carrying case portion 405d, may have hexagonal cross-sections (FIG. 4, panel D). The first carrying case portion, canister, and second carrying case portion also may have octagonal or pentagonal cross-sections.

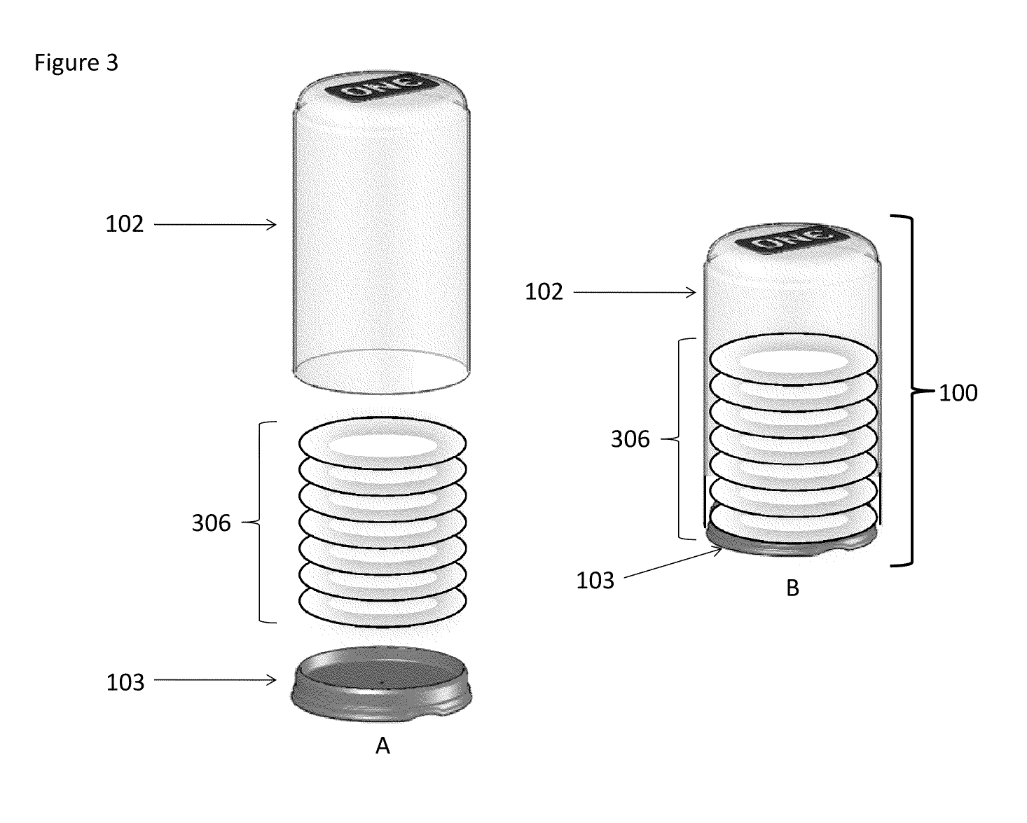

As illustrated in FIG. 3, the canister 102 may store one (e.g. 6, 8, 12, 24 or any other number) prophylactic devices, such as condoms 306. The open end of the canister 102 may be closed with either the lid 103 (FIG. 3) or the second carrying case portion 105. The canister 102 has a primary axis and is shaped to store the prophylactic devices stacked along the primary axis. For example, and as illustrated in FIG. 3, for condoms 306 having circular packages, the canister 102 is cylindrical with a radius slightly larger than the radius of the packaged condom 306. For example, the canister 102 may have a diameter of about 2.5 to about 3.0 inches. For square-packaged condoms, the canister 402c (FIG. 4, Panel C) may be a rectangular prism having a square cross-section that is slightly larger than the square-shape of the condom package. For example, each side of the cross-section of the canister 402c may be between about 2.5 and about 3.00 inches. The canister may store at least five times the number of individual prophylactic devices that can fit in the carrying case. In other embodiments, the canister may be sized such that it holds two times, three times, four times, or even up to ten or more times the number of condoms that fit in the carrying case. Preferably, the canister may be between about 4 to about 24. inches long. As illustrated in FIGS. 1-3, the canister 102 may be transparent, e.g., formed from a transparent plastic. Alternatively, the canister also may be opaque.

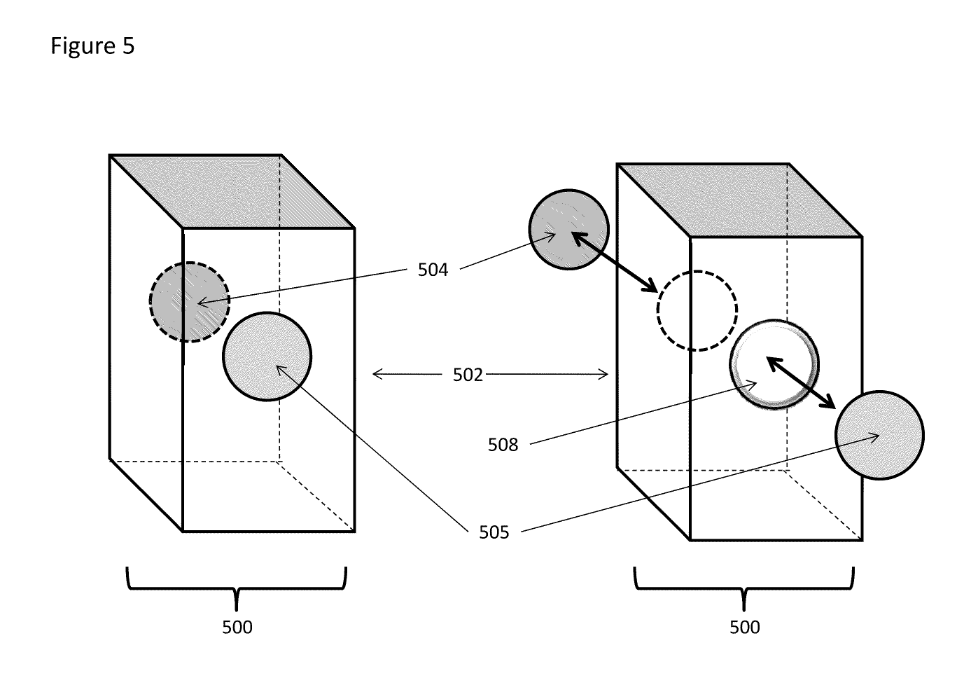

The first carrying case portion 104 and the second carrying case portion 105 may be attached to any surface of the canister 102. For example, in FIG. 2 the first carrying case portion 104 and the second carrying case portion 105 are attached to the open end and closed end of the canister, respectively. As illustrated in FIG. 5, either the first carrying case portion 504 or the second carrying case portion 505, or both, may be fitted securely to plastic protrusions 508 extending from sides of the canister 502 or into recesses formed in the sides of the container 502. In each case, the first and second carrying case portions may be removed from the canister 502 and fitted together to form a carrying case 509.

As illustrated in FIG. 6, the container 100 may include a first carrying case portion 104 and a second carrying case portion 105 that may be fitted together to form a carrying case 609 that can store one or multiple prophylactic devices, such as condoms 306. The carrying case 609 may store at least two times, three times, four times, five times or even up to ten or fewer times the number of condoms that fit in the canister. The carrying case also may be pocket-sized.

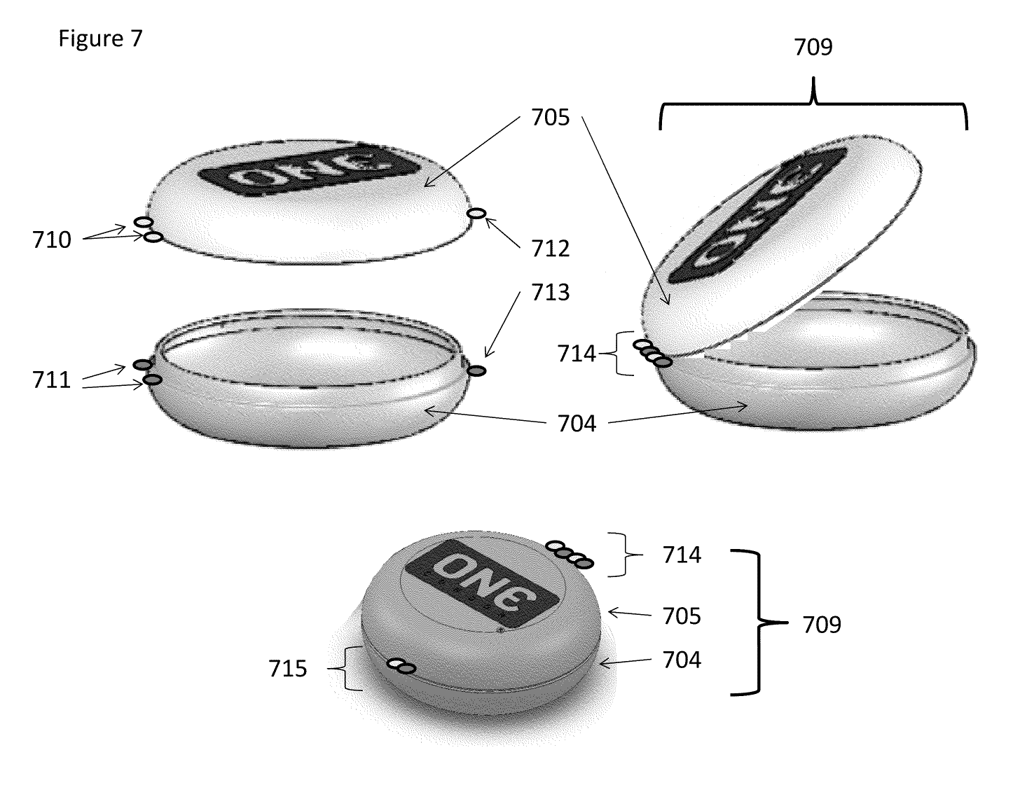

As illustrated in FIG. 7, the container 100 may include a first carrying case portion 704 which has a first hinge element 711 and a first clasp element 713, and a second carrying case portion 705 which has a second hinge element 710 and a clasp element 712. The first hinge element 711 and the second hinge element 710 may be snapped easily together to form a hinged carrying case 709 that may be opened and closed by means of the hinge 714. The first clasp element 711 and the second clasp element 712 may also be fitted together to form a clasp 715 that allows the carrying case 709 to remain closed during storage but easily opened when desired.

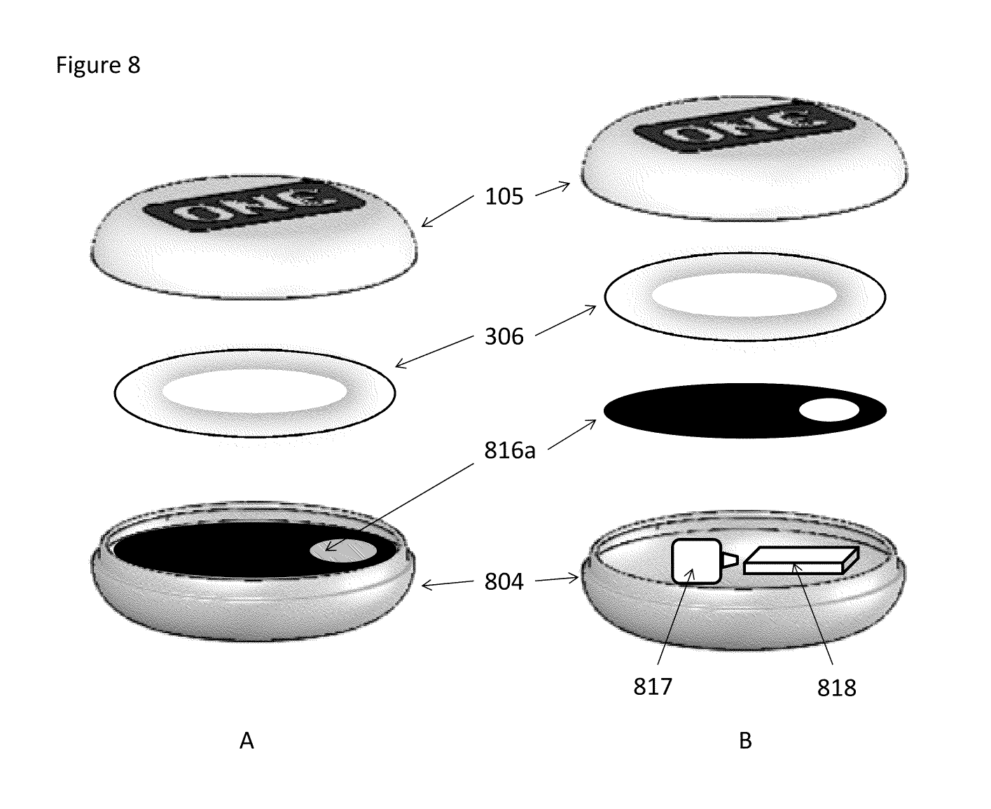

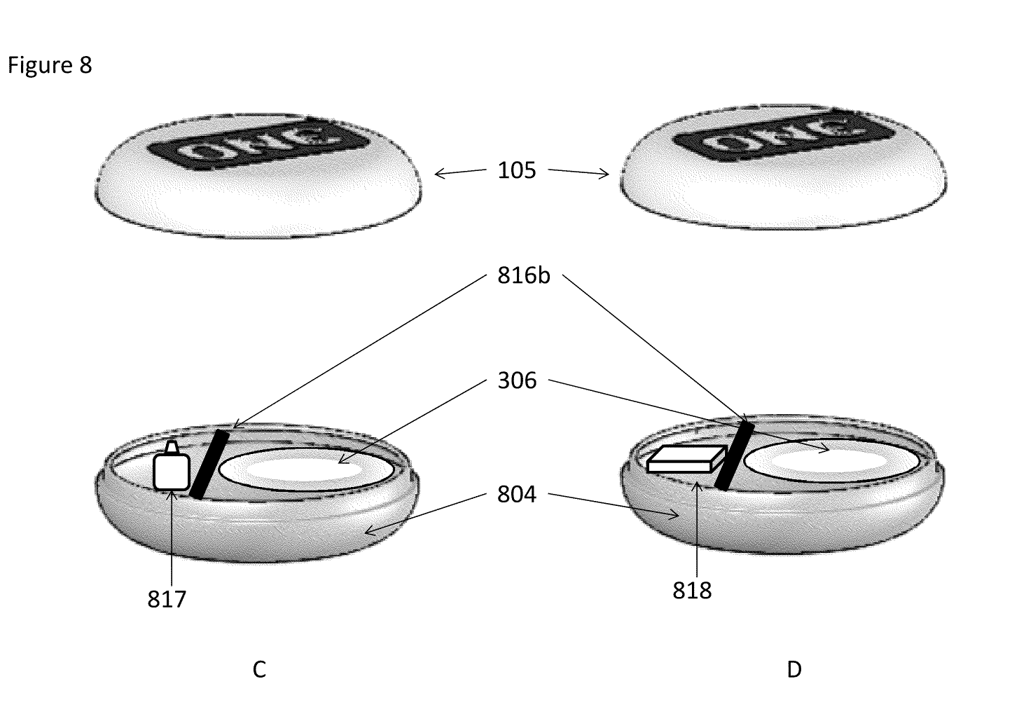

The container 100 may include either a first carrying case portion 804 or a second carrying case portion 105 having a divider 816 that separates either the first carrying case portion 804 or the second carrying case portion 105, or both, into two compartments. As illustrated in FIG. 8, panels A-B, the divider 816a may separate the first carrying case portion 804 into a top and bottom compartment. The divider 816 could be removable. The top or bottom compartment may store one or several condoms 306, while the other compartment may store an object that is not a condom, such as a package of lubricant 817, a packaged dental dam 818, or one or several birth control pills. As illustrated in FIG. 8, panels C-D, the divider 816b may separate the first carrying case portion 804, or the second carrying case portion 105, into two side compartments, in which a condom 306 is stored in one compartment, and an object or objects, such as a lubricant 817, a packaged dental dam 818, or one or several birth control pills, is stored in the other compartment.

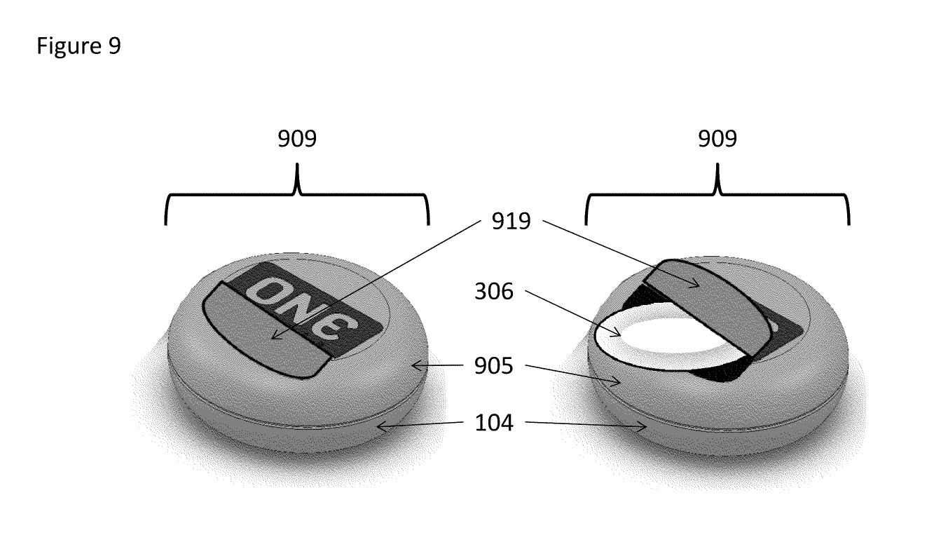

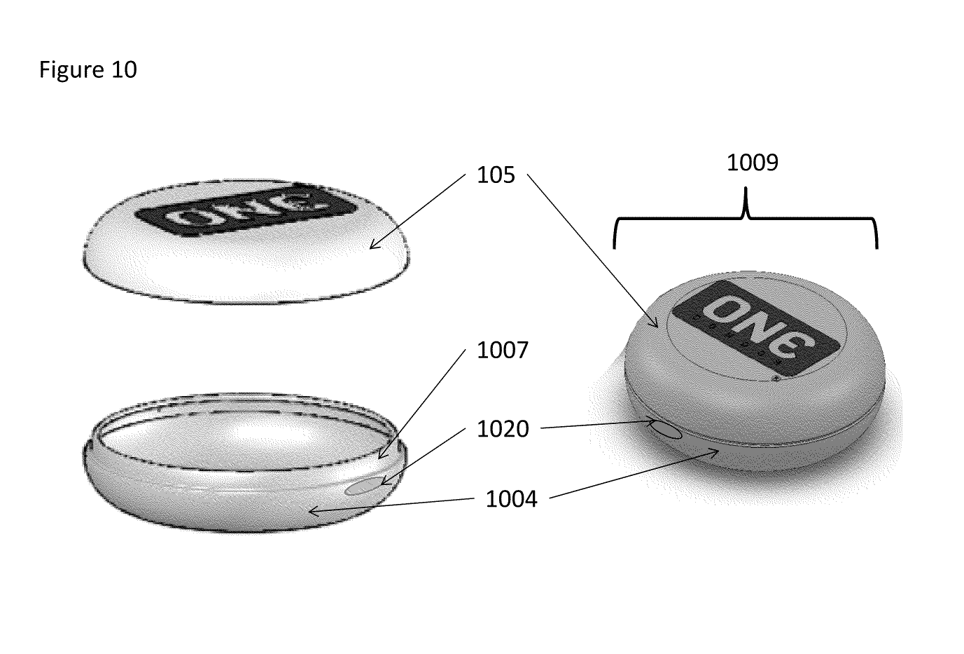

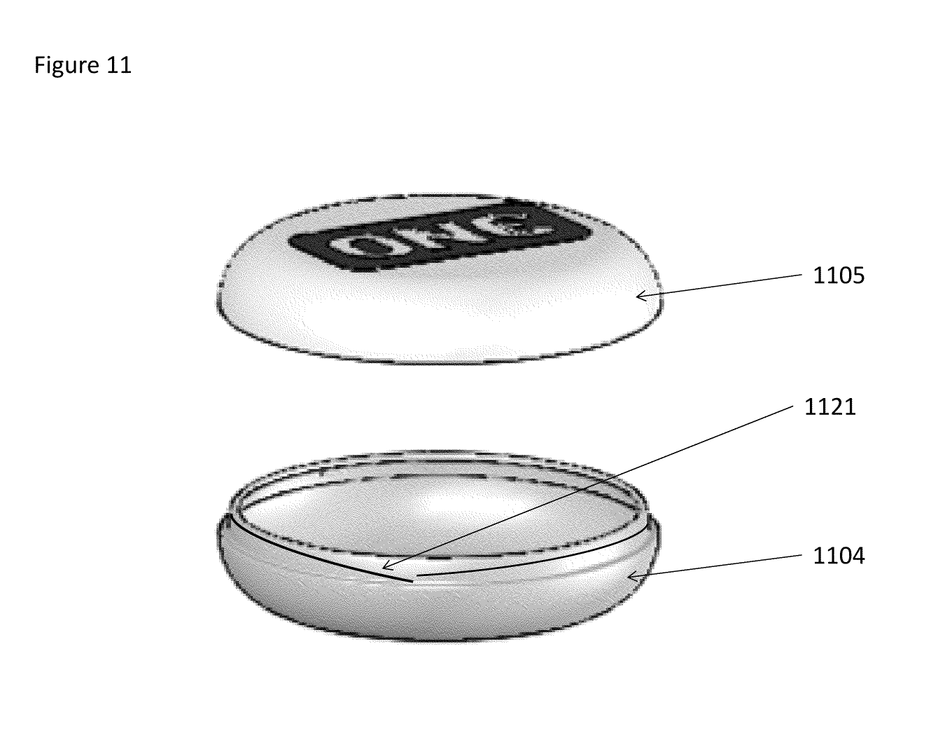

As illustrated in FIG. 9, the container 100 has a second carrying case portion 905 having a hinged door 919 which may be opened to access a condom held within the carrying case 909. As illustrated in FIG. 10, the container may include a first carrying case portion 1004 having a lip 1007 over which the second carrying case portion 105 may slide in order to engage the second carrying case portion 105 to the first carrying case portion 1004. As illustrated in FIG. 10, the container 100 may include a first carrying case portion 1004 or a second carrying case portion 105, or both the first carrying case portion 1004 and the second carrying case portion 105, having an indentation 1020 just below, or an indentation just above, the region at which the first carrying case portion and the second carrying case portion are joined together. The indentation or indentations may be used to facilitate a user to apply a separation pressure to disengage the first carrying case portion from the second carrying case portion. As illustrated in FIG. 11, the container may have a second carrying case portion 1104 having a lip which has threading 1121 over which the second carrying case portion 1105 may be twisted in order to join the second carrying case portion 1105 to the first carrying case portion 1104.

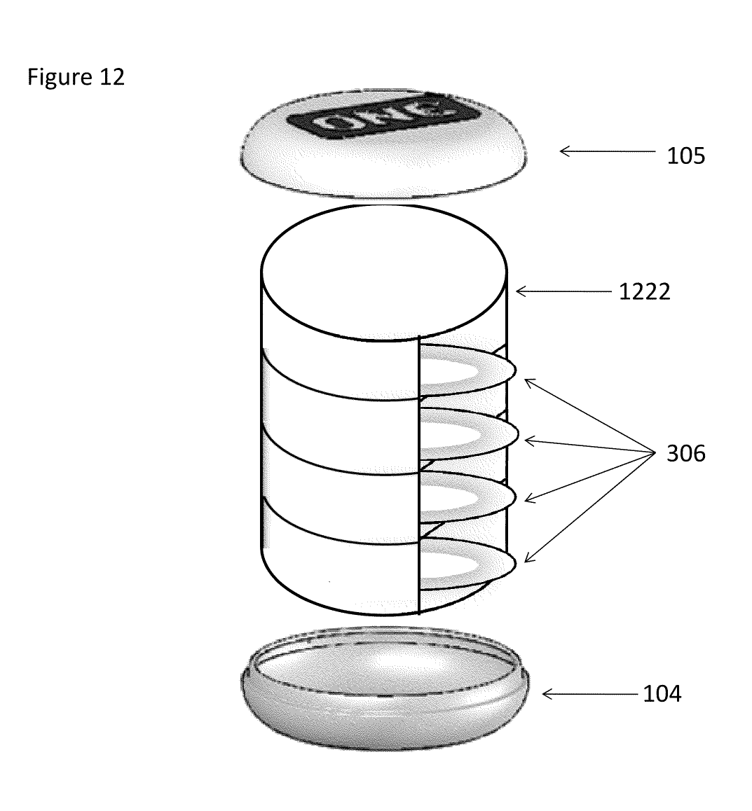

As illustrated in FIG. 12, the carrying case 609 of the container 100 may include a collapsible series of pockets 1222. At least one prophylactic device, such as a condom 306, may fit into each pocket of the collapsible series of pockets 1222. The collapsible series of pockets 1222 may be attached to the first carrying case portion 104, the second carrying case portion 105, or both. The collapsible series of pockets 1222 also may be removable. The series of pockets may be arranged in a fan-out style, similar to the series of pockets used for storing pictures in a wallet. Alternatively, as illustrated in FIG. 12, the series of pockets 1222 may be arranged in an accordion style.

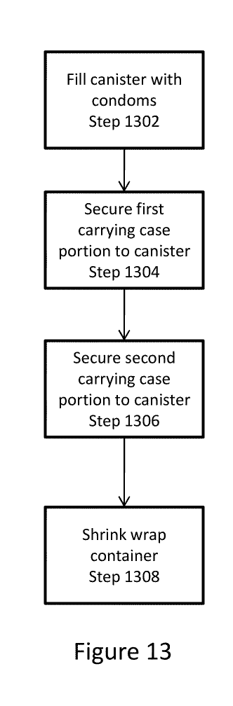

FIG. 13 is a flow chart of a method for packing condoms. This method includes the step of filling a canister having an open end and a closed end with multiple condoms (step 1302). Next, a first carrying case portion is securely but removably fixed (step 1304) to the open end of the canister and the second carrying case portion is securely but removably fixed to the closed end of the canister (step 1306). The carrying case portions used in this method are also configured to engage each other to form a carrying case. At least one, but fewer than all, of the condoms filled into the canister may be stored in the carrying case. Finally, the method includes the step of shrink-wrapping the canister along with the first and second carrying case portions securely but removably attached to it (step 1308).

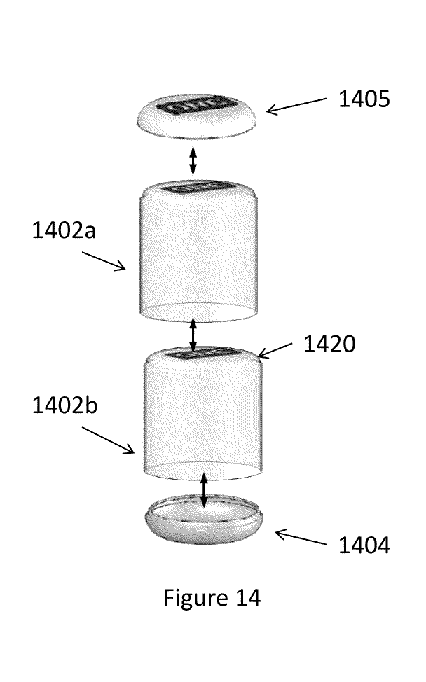

FIG. 14 depicts an alternative embodiment of the container, which includes two canisters 1402a and 1402b, according to an illustrative embodiment of the invention. Each container includes an open end and a closed end. Two carrying case portions 1404 and 1405 are sized to securely, but removably engage the open ends of the canisters 1402a and 1402b and the closed ends of the canisters 1402a and 1402b, respectively. The closed ends of the canisters 1402a and 1402b narrow to form a shoulder portion 1420. The shoulder portions are sized such that they can securely, but removably engage both the open end of the other canister 1402a or 1402b and the second carrying case portion 1405. For use in stores, the first and second canisters 1402a and 1402b can be filled with different types of products, for example and without limitation, different types of condoms in each canister 1402a and 1402b, condoms in one canister 1402a and packaged lubricant or dental dams in the other canister, etc. The two canisters 1402a and 1402b, engaged with each other and the first and second carry case portions 1404 and 1405 are then shrink wrapped together as a single package.

In alternative embodiments a single canister can be divided into multiple compartments with a horizontal divider, separating the canister into upper and lower compartments, or with a vertical divider, running the length of the canister.

The invention may be embodied in other specific forms without departing from the spirit or essential characteristics thereof. The foregoing embodiments are therefore to be considered in all respects illustrative, rather than limiting of the invention.

What is claimed is:

1. A container for holding at least one prophylactic device comprising:
a canister sized to store a plurality of prophylactic devices, wherein the canister has first and second ends; and
first and a second carrying case portions, wherein
i) the first carrying case portion is configured to engage the second carrying case portion to form a carrying case for at least one, but fewer than all, of the prophylactic devices from the canister; and
ii) the canister, the first carrying case portion, and the second carrying case portion are configured such that the first and second carrying case portions can be securely, but removably coupled to the canister.

2. The container of claim 1, comprising a lid configured to engage both the first end of the canister and the first carrying case portion, thereby securely but removably coupling the first carrying case portion to the canister.

3. The container of claim 2, wherein the second end of the canister is closed, and the second carrying case portion is configured to engage the second, closed end of the canister.

4. The container of claim 1, wherein the first end of the canister is open, and the first carrying case portion is configured to engage and at least partially close the first, open end of the canister.

5. The container of claim 1, wherein the second end of the canister is closed, and the second carrying case portion is configured to engage the second, closed end of the canister.

6. The carrying case of claim 1, the first and second carrying case portions comprise corresponding threading such that the first and second carrying case portions can be screwed together.

7. The carrying case of claim 1, wherein the first and the second carrying case portion engage to form a hinge at a first position and a clasp at a second position.

8. The container of claim 1, wherein the prophylactic device is a condom.

9. The container of claim 8, wherein the container further comprises a divider configured for insertion into the carrying case to separate the carrying case into a first portion sized to hold a condom and a second portion sized to hold a second object.

10. The container of claim 9, wherein the second object is a package of lubricant or a packaged dental dam.

11. The container of claim 1, wherein the canister has circular, hexagonal, rectangular or oval cross-section.

12. The container of claim 1, wherein the container further comprises a plurality of pockets coupled to one another, wherein each of the pockets is sized to accommodate a condom, and the plurality of pockets is sized to fit within the carrying case.

13. The container of claim 1, wherein the first carrying case portion comprises a hinged portion capable of being opened in order to access a prophylactic device held within the carrying case.

14. The container of claim 1, wherein the first carrying case portion comprises an indentation at a location that, when the first carrying case portion is engaged with the second carrying case portion, facilitates a user applying a separation pressure to the second carrying case portion to disengage the first carrying case portion from the second carrying case portion.

15. The container of claim 1, wherein the first carrying case portion comprises a lip over which the second carrying case portion slides in order to engage the first carrying case portion.

16. The container of claim 1, wherein the canister has a primary axis and is shaped to store the plurality of prophylactic devices stacked along the primary axis.

17. The container of claim 1, wherein the canister stores at least five times the number of individual prophylactic devices that can fit in the carrying case.

18. The container of claim 1, wherein the carrying case is pocket-sized.

19. The container of claim 1, wherein the canister is transparent.

20. A method for packaging condoms comprising:
providing a canister sized to store a plurality of condoms, wherein the canister has an first end and a second end;
inserting a plurality of condoms into the canister;
providing first and second carrying case portions, wherein the first carrying case portion is configured to engage the second carrying case portion to form a carrying case for at least one, but fewer than all, of the condoms that were inserted into the canister;
securely but removably coupling the first carrying case portion to the first end of the canister; and
securely but removably coupling the second carrying case portion to the second end of canister.

21. The method of packaging condoms of claim 20, comprising shrink-wrapping the canister with the first and second carrying case portions securely but removably coupled to it.
